(19) United States
(12) Patent Application Publication
(10) Pub. No.: US 2021/0015980 A1
(43) Pub. Date: Jan. 21, 2021

(54) DUAL-CHAMBER SUCTION FILTERING DEVICE AND FILTERING METHOD

(71) Applicant: BEIJING LONGFU HOSPITAL, Beijing (CN)

(72) Inventors: Yonglei LI, Beijing (CN); Jie DING, Beijing (CN); Tongqing CUI, Beijing (CN); Wei LI, Beijing (CN); Chao GUI, Beijing (CN); Zhe LIU, Beijing (CN); Ting LV, Beijing (CN); Fangxiang LI, Beijing (CN); Qiang XIE, Beijing (CN); Hongchen WANG, Beijing (CN)

(73) Assignee: BEIJING LONGFU HOSPITAL, Beijing (CN)

(21) Appl. No.: 16/982,953

(22) PCT Filed: Mar. 1, 2019
(86) PCT No.: PCT/CN2019/076684

§ 371 Date: Sep. 21, 2020

(30) Foreign Application Priority Data
Mar. 21, 2018 (CN) 201810233359.1

(51) Int. Cl. A61M 1/00; A61M 39/24

(52) U.S. Cl. CPC A61M 1/0056; A61M 1/0011; A61M 2205/75; A61M 39/24; A61M 1/0094

(57) ABSTRACT

A dual-chamber suction filtering device and filtering method, belonging to a surgical waste liquid collecting device, and solving the problem in the prior art that drainage fluid of an endoscope shaver system and a plasma surgical system is easily contaminated and cannot be separated in sections. The dual-chamber suction filtering device comprises a dual-chamber one-way valve (1), a switching valve (4), and a filtering branch (2) and a direct-passing main path (3) which are connected to each other in parallel, the water outlet of the dual-chamber one-way valve (1) being connected to the water inlet of the filtering branch (2) and the water inlet of direct-passing main path (3); the switching valve (4) is provided on the joint between the water inlet of the filtering branch (2) and the water inlet of direct-passing main path (3); the dual-chamber one-way valve (1) has a first drainage fluid system (5) setting, a second drainage fluid system (6) setting, and a dual-drainage fluid system (5, 6) setting, and the switching valve (4) has a filtering setting and a direct-passing setting. The dual-chamber suction filtering device and filtering method can be used for collecting and filtering drainage fluid.

### TECHNICAL FIELD

The invention relates to a surgical waste liquid collecting device, in particular to a dual-chamber suction filtering device and a filtering method.

### BACKGROUND ART

There will be tissue fluid and soft tissue debris in the drainage fluid of an endoscope shaver system and a plasma surgery system in the operation process, which can be used for pathological tests or for histology and molecular biology research in scientific research experiments.

In the prior art, the drainage fluid of the endoscope shaver system and plasma surgical system is directly connected to a waste liquid bottle via a negative pressure suction device.

However, it is easy to contaminate the drainage fluid in the collection process of the drainage fluid as there is no complete closed-chain sterile environment, so that tissue fluid and soft tissue debris in the drainage fluid cannot be used for subsequent tests and experiments. Meanwhile, due to the fact that the drainage fluid cannot be selectively separated in sections, resulting excess drainage fluid in the waste liquid bottle, so that extraction cannot be effectively conducted, and subsequent tests and experiments may be influenced.

### SUMMARY OF THE INVENTION

In view of the above analysis, the present application aims to provide a dual-chamber suction filtering device and a filtering method, solving the problem in the prior art that drainage fluid of an endoscope shaver system and a plasma surgical system is easily contaminated and cannot be separated in sections.

The purpose of the present application is mainly achieved by the following technical scheme:

The present application provides a dual-chamber suction filtering device comprising a dual-chamber one-way valve, a switching valve, and a filtering branch and a direct-passing main path which are connected to each other in parallel; the water outlet of the dual-chamber one-way valve is connected to the water inlet of the filtering branch and the water inlet of direct-passing main path, respectively; the switching valve is provided on the joint between the water inlet of the filtering branch and the water inlet of direct-passing main path; the dual-chamber one-way valve has a first drainage fluid system setting, a second drainage fluid system setting and a dual-drainage fluid system setting, and the switching valve has a filtering setting and a direct-passing setting; and the water inlet of the dual-chamber one-way valve is connected to the first drainage fluid system and/or the second drainage fluid system when in use, respectively.

Furthermore, the switching valve also has a fluid stop setting.

Furthermore, in the dual-chamber one-way valve, an included angle connected to the water inlet of the first drainage fluid system and the water inlet of the second drainage fluid system is 60°, an included angle connected to the water inlet and the water outlet of the first drainage fluid system is 120°, and an included angle connected to the water inlet and the water outlet of the second drainage fluid system is 120°.

Furthermore, a stirring member is arranged on one side of the switching valve close to the dual-chamber one-way valve.

Furthermore, a spiral structure is arranged on an inner wall of a connecting pipeline between the dual-chamber one-way valve and the switching valve.

Furthermore, a path length of the direct-passing main path is less than that of the filtering branch.

Furthermore, the filtering branch is detachably connected in parallel to the two ends of the direct-passing main path via connection joints.

Furthermore, the filtering branch comprises a filtering barrel and a filtering member arranged in the filtering barrel.

The present application also provides a dual-chamber suction filtering method, which adopts the above-mentioned dual-chamber suction filtering device and comprises the following steps:

Step S1: the water inlet of the dual-chamber one-way valve is connected to the first drainage fluid system and the second drainage fluid system respectively, and the water outlet of the filtering branch and the water outlet of the direct-passing main path are connected to a fluid collection bottle;

Step S2: when it is necessary to collect and filter the drainage fluid to be detected in the first drainage fluid system and the second drainage fluid system at the same time, the dual-chamber one-way valve is switched to a dual-drainage fluid system setting, both of the first drainage fluid system and the second drainage fluid system are connected to the water inlet of the dual-chamber one-way valve, and the switching valve is switched to a filtering setting; the drainage fluid in the first drainage fluid system and the second drainage fluid system flows through the dual-chamber one-way valve and the filtering branch in turn and then flows into the fluid collection bottle, and soft tissue debris may be filtered and retained in the filtering branch when the drainage fluid flows through the filtering branch;

when it is necessary to collect and filter the drainage fluid to be detected in the first drainage fluid system, the dual-chamber one-way valve is switched to a first drainage fluid system connection setting, and the switching valve is switched to a filtering setting; the drainage fluid in the first drainage fluid system flows through the dual-chamber one-way valve and the filtering branch in turn and then flows into the fluid collection bottle, and soft tissue debris may be filtered and retained in the filtering branch when the drainage fluid flows through the filtering branch;

when it is necessary to collect and filter the drainage fluid to be detected in the second drainage fluid system, the dual-chamber one-way valve is switched to a second drainage fluid system setting, and the switching valve is switched to a filtering setting; the drainage fluid in the second drainage fluid system flows through the dual-chamber one-way valve and the filtering branch in turn and then flows into the fluid collection bottle, and soft tissue debris may be filtered and retained in the filtering branch when the drainage fluid flows through the filtering branch; when there is no need to collect and filter the drainage fluid in the first drainage fluid system and/or the second drainage fluid system, the dual-chamber one-way valve is switched to a dual-drainage fluid system setting and the switching valve is switched to a direct-passing setting, the drainage fluid in the first drainage fluid system and/or the second drainage fluid system flows into the fluid collection bottle via the direct-passing main path.

Compared with the prior art, the present application has the following beneficial effects:

a) the dual-chamber suction filtering device provided by the present application is provided with a dual-chamber one-way valve by which the water outlet of the dual-chamber one-way valve may be controlled to be connected to or disconnected from the first drainage fluid system and the second drainage fluid system, so that a certain section of drainage fluid to be detected in the first drainage fluid system and the second drainage fluid system may be selectively collected, and the dilution of the drainage fluid to be detected may be avoided;

b) the dual-chamber suction filtering device provided by the present application is also provided with a filtering branch which is connected in parallel to the direct-passing main path, the water outlet of the dual-chamber one-way valve may be controlled to be connected to or disconnected from the filtering branch and the direct-passing main path by the switching valve, when the drainage fluid needs to be filtered, the switching valve is switched to a filtering setting, and the drainage fluid flowing out of the dual-chamber one-way valve flows through the filtering branch and then flows into the fluid collection bottle, thereby filtering and separating the soft tissue debris in the drainage fluid to be detected;

c) the dual-chamber suction filtering device provided by the present application is a set of independent drainage fluid filtering device, which can ensure that the entire process of filtration and collection of drainage fluid is in a sterile environment, thereby reducing the possibility of contamination in drainage fluid.

Other features and advantages of this application will be described in the following description, and part of them will become obvious from the description, or be understood by implementing this application. The purpose and other advantages of this application can be implemented and obtained through the structure specified in the written description, claims, and drawings.

### BRIEF DESCRIPTION OF THE DRAWINGS

The drawings, in which like reference numerals refer to like parts throughout, are only for the purpose of illustrating specific Examples and are not to be considered as limitation of the application.

FIG. 1 is a schematic view showing the structure of a dual-chamber suction filtering device provided in Example 1 of the present application;

FIG. 2 is a schematic view showing the structure of a dual-chamber suction filtering device in a state of collecting and filtering the drainage fluid in a first drainage fluid system and a second drainage fluid system simultaneously provided in Example 1 of the present application;

FIG. 3 is a schematic view showing the structure of a dual-chamber suction filtering device in a state of collecting and filtering the drainage fluid in a first drainage fluid system provided in Example 1 of the present application;

FIG. 4 is a schematic view showing the structure of a dual-chamber suction filtering device in a state of collecting and filtering the drainage fluid in a second drainage fluid system provided in Example 1 of the present application;

FIG. 5 is a schematic view showing the structure of a dual-chamber suction filtering device in a state of unfiltering the drainage fluid provided in Example 1 of the present application; In the figure, the direction of the arrow indicates the flow direction of the drainage fluid.

### REFERENCE NUMERALS

1—dual-chamber one-way valve; 101—valve core; 102 valve sleeve; 2—filtering branch; 201—filtering barrel; 202—filtering member; 3—direct—passing main path; 4—switching valve; 5—first drainage fluid system; 6—second drainage fluid system; 7—fluid collection bottle; 8—stirring member; 9—spiral structure; 10—stopper; 11—connection joint.

### DETAILED DESCRIPTION OF THE INVENTION

The preferred Examples of the present application are described in detail below with reference to the accompanying drawings, which form a part hereof, and which together with the Examples of the application serve to explain the principles of the application.

### Example 1

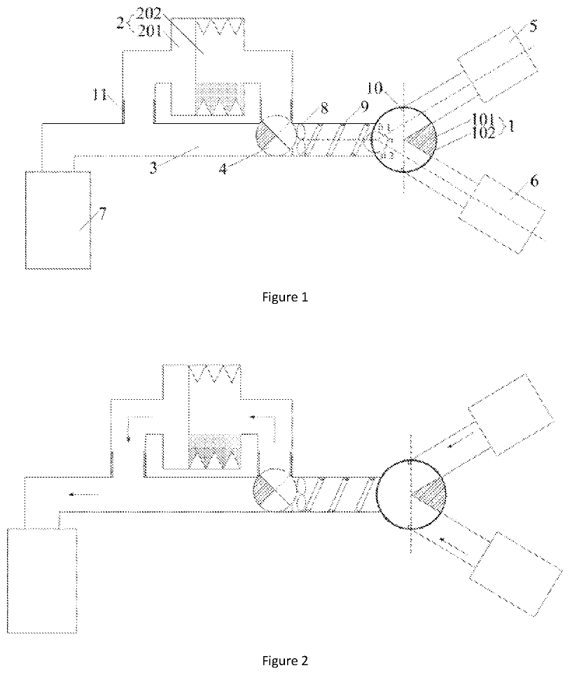

As shown in FIGS. 1-5, the present application provides a dual-chamber suction filtering device comprising a dual-chamber one-way valve 1, and a filtering branch 2 and a direct-passing main path 3 which are connected to each other in parallel; the water outlet of the dual-chamber one-way valve 1 is connected to the water inlet of the filtering branch 2 and the water inlet of direct-passing main path 3, respectively; a switching valve 4 is provided on the joint between the water inlet of the filtering branch 2 and the water inlet of direct-passing main path 3; the dual-chamber one-way valve 1 has a first drainage fluid system setting, a second drainage fluid system setting and a dual-drainage fluid system setting, and the switching valve 4 has a filtering setting and a direct-passing setting.

It should be noted that the first drainage fluid system 5 and the second drainage fluid system 6 may be an endoscope shaver system and a plasma surgical system, or other systems requiring the collection and filtration of drainage fluid therefrom, which are not limited here.

The water inlet of the dual-chamber one-way valve 1 is connected to the first drainage fluid system 5 and the second drainage fluid system 6 respectively, and the water outlet of the filtering branch 2 and the water outlet of the direct-passing main path 3 are connected to a fluid collection bottle 7 when in use;

when it is necessary to collect and filter certain section of drainage fluid to be detected in the first drainage fluid system 5 and the second drainage fluid system 6 at the same time, as shown in FIG. 2, the dual-chamber one-way valve 1 is switched to a dual-drainage fluid system setting, and both of the first drainage fluid system 5 and the second drainage fluid system 6 are connected to the water inlet of the dual-chamber one-way valve 1; the switching valve 4 is switched to a filtering setting, the water outlet of the dual-chamber one-way valve 1 is connected to the fluid collection bottle 7 via the filtering branch 2, and the water outlet of the dual-chamber one-way valve 1 is disconnected from the direct-passing main path 3; the drainage fluid in the first drainage fluid system 5 and the second drainage fluid system 6 flows through the dual-chamber one-way valve 1 and the filtering branch 2 in turn and then flows into the fluid collection bottle 7, and soft tissue debris may be filtered and retained in the filtering branch 2 when the drainage fluid flows through the filtering branch 2, thereby separating the soft tissue debris from the drainage fluid; after the collection of the drainage fluid in this section is completed, the collection bottle 7 containing the drainage fluid to be detected may be removed to obtain the uncontaminated and diluted drainage fluid to be detected in the first drainage fluid system 5 and the second drainage fluid system 6.

When it is necessary to collect and filter certain section of drainage fluid to be detected in the first drainage fluid system 5, as shown in FIG. 3, the dual-chamber one-way valve 1 is switched to a first drainage fluid system setting, the first drainage fluid system 5 is connected to the water inlet of the dual-chamber one-way valve 1, and the second drainage fluid system 6 is disconnected from the water inlet of the dual-chamber one-way valve 1; the switching valve 4 is switched to a filtering setting, the water outlet of the dual-chamber one-way valve 1 is connected to the fluid collection bottle 7 via the filtering branch 2, and the water outlet of the dual-chamber one-way valve 1 is disconnected from the direct-passing main path 3; the drainage fluid in the first drainage fluid system 5 flows through the dual-chamber one-way valve 1 and the filtering branch 2 in turn and then flows into the fluid collection bottle 7, and soft tissue debris may be filtered and retained in the filtering branch 2 when the drainage fluid flows through the filtering branch 2, thereby separating the soft tissue debris from the drainage fluid; after the collection of the drainage fluid to be detected is completed, the collection bottle 7 containing the drainage fluid of this section may be removed to obtain the uncontaminated and diluted drainage fluid to be detected in the first drainage fluid system 5.

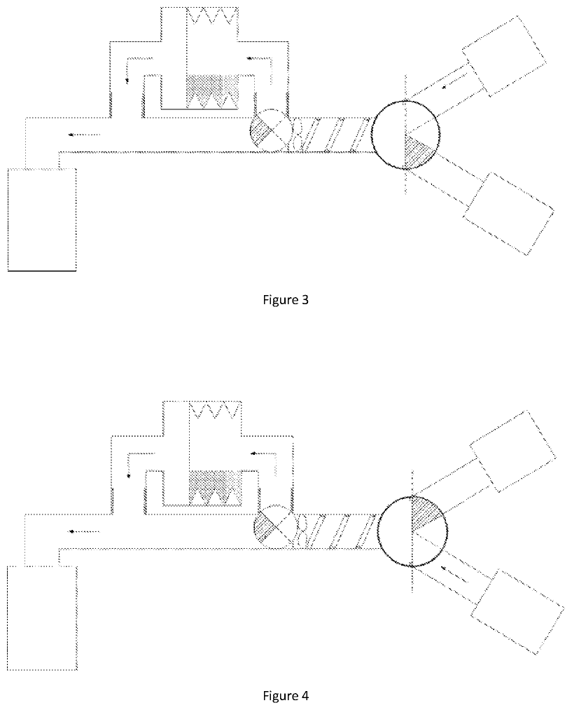

Similarly, when it is necessary to collect and filter drainage fluid to be detected in the second drainage fluid system 6, as shown in FIG. 4, the dual-chamber one-way valve 1 is switched to a second drainage fluid system setting, the second drainage fluid system 6 is connected to the water inlet of the dual-chamber one-way valve 1, and the first drainage fluid system 5 is disconnected from the water inlet of the dual-chamber one-way valve 1; the switching valve 4 is switched to a filtering setting, the water outlet of the dual-chamber one-way valve 1 is connected to the fluid collection bottle 7 via the filtering branch 2, and the water outlet of the dual-chamber one-way valve 1 is disconnected from the direct-passing main path 3; the drainage fluid in the second drainage fluid system 6 flows through the dual-chamber one-way valve 1 and the filtering branch 2 in turn and then flows into the fluid collection bottle 7, and soft tissue debris may be filtered and retained in the filtering branch 2 when the drainage fluid flows through the filtering branch 2, thereby separating the soft tissue debris from the drainage fluid; after the collection of the drainage fluid of this section is completed, the collection bottle 7 containing the drainage fluid to be detected may be removed to obtain the uncontaminated and diluted drainage fluid to be detected in the second drainage fluid system 6.

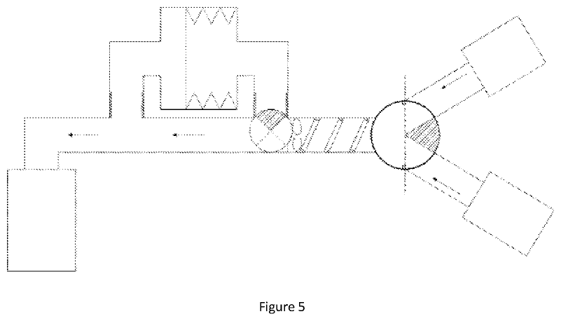

When there is no need to collect and filter the drainage fluid in the first drainage fluid system 5 and/or the second drainage fluid system 6, as shown in FIG. 5, the dual-chamber one-way valve 1 is switched to at a dual-drainage fluid system setting, the switching valve 4 is switched to a direct-passing setting, the water outlet of the dual-chamber one-way valve 1 is connected to the fluid collection bottle 7 via the direct-passing main path 3, and the water outlet of the dual-chamber one-way valve 1 is disconnected from the filtering branch path 2, and the drainage fluid in the first drainage fluid system 5 and/or the second drainage fluid system 6 flows directly into the collection bottle 7 without passing through the filtering branch 2.

Compared with the prior art, the dual-chamber suction filtering device provided by the present application is provided with a dual-chamber one-way valve 1 by which the water outlet of the dual-chamber one-way valve may be controlled to be connected to or disconnected from the first drainage fluid system 5 and the second drainage fluid system 6, so that a certain section of drainage fluid to be detected in the first drainage fluid system 5 and the second drainage fluid system 6 may be selectively collected, and the dilution of the drainage fluid to be detected may be avoided. Meanwhile, the above-mentioned dual-chamber suction filtering device is also provided with a filtering branch 2 which is connected in parallel to the direct-passing main path 3, the water outlet of the dual-chamber one-way valve 1 may be controlled to be connected to or disconnected from the filtering branch 2 and the direct-passing main path 3 by the switching valve 4, when the drainage fluid needed to be filtered, the switching valve 4 is switched to a filtering setting, and the drainage fluid flowing out of the dual-chamber one-way valve 1 flows through the filtering branch 2 and then flows into the fluid collection bottle 7, thereby filtering and separating the soft tissue debris in the drainage fluid to be detected.

In addition, the above-mentioned dual-chamber suction filtering device is a set of independent special drainage fluid filtering device, which can ensure that the entire process of filtration and collection of drainage fluid is in a sterile environment, thereby reducing the possibility of contamination in drainage fluid.

As in the process of collecting the drainage fluid to be detected, the collection bottle 7 containing the drainage fluid to be detected needed to be removed and replaced with other collection bottles 7. The above-mentioned switching valve 4 also has a fluid stop setting to prevent the drainage fluid from continuously flowing out and contaminating the ground or other objects during the replacement. When the fluid collection bottle 7 needed to be replaced, the switching valve 4 may be temporarily switched to the fluid stop setting, and the dual-chamber one-way valve 1 is disconnected from the filtering branch 2 and the direct-passing main path 3 at the moment, so that drainage fluid could not flow out of the drainage fluid filtering device, thus avoiding the contamination of the ground or other objects by the drainage fluid in the process of replacing the liquid collecting bottle 7.

For convenience in installation, in the above-mentioned dual-chamber one-way valve 1, an included angle α connected to the water inlet of the first drainage fluid system 5 and the water inlet of the second drainage fluid system 6 is 60°, an included angle β1 connected to the water inlet and the water outlet of the first drainage fluid system is 120°, and an included angle β2 connected to the water inlet and the water outlet of the second drainage fluid system is 120°. This arrangement can ensure that the first drainage fluid system 5 and the second drainage fluid system 6 are located at one side of the dual-chamber one-way valve 1, and the filter branch 2, the direct-passing main path 3 and the fluid collection bottle 7 are located at the other side of the dual-chamber one-way valve 1. In the process of assembling the drainage fluid filtering device, an operator may connect it to the end of the water inlet firstly and then to the end of the water outlet, which conforms to the operating habits. Meanwhile, due to the fact that the angle between the two water inlets is different from the angle between the water inlets and the water outlets, the operator can accurately determine the water inlet and the water outlet according to the different angles, thereby avoiding installation errors. In addition, this arrangement can also appropriately reduce the deposition of soft tissue debris in the drainage fluid in the corners between the dual-chamber one-way valve and the side wall of the connecting pipeline.

In order to prevent soft tissue debris in the drainage fluid from being deposited in the corner between the switching valve 4 and the side wall of the connecting pipeline, a stirring member 8 such as a stirring rod or a stirring paddle or the like may be arranged on the side of the switching valve 4 close to the dual-chamber one-way valve 1. Therefore, when the drainage fluid flows through the stirring member 8 which would rotate under the action of the thrust force generated by the flow of the drainage fluid, so that the drainage fluid on the side of the switching valve 4 close to the dual-chamber one-way valve 1 is disturbed, thus preventing the soft tissue debris in the drainage fluid from being deposited in the corner between the switching valve 4 and the side wall of the connecting pipeline.

Similarly, in order to prevent soft tissue debris in the drainage fluid from being deposited in the corner between the switching valve 4 and the side wall of the connecting pipeline, a spiral structure 9 may also be provided on the inner wall of the connecting pipeline between the dual-chamber one-way valve 1 and the switching valve 4; the drainage fluid flows into the spiral connecting pipeline, leading to larger disturbance of the drainage fluid, thus preventing the soft tissue debris in the drainage fluid from being deposited in the corner between the switching valve 4 and the side wall of the connecting pipeline.

It will be appreciated that a combination of the stirring member 8 and the spiral structure 9 may be used to further increase the turbulence of the drainage fluid, but it should be noted that the rotation direction of the stirring member 8 should be the same as the spiral direction of the spiral structure 9, otherwise, it will affect the flow of drainage fluid in the connecting pipeline. In particular, the spiral structure 9 may be a spiral groove or a spiral protrusion, the groove or protrusion being connected to the inner wall of the connecting pipeline in an arc smooth transition.

As to the structure of the dual-chamber one-way valve 1, in particular, the dual-chamber one-way valve 1 comprises a valve sleeve 102 and a valve core 101 arranged inside the valve sleeve 102, the valve core 101 and the valve sleeve 102 are mutually cooperated by which the dual-chamber one-way valve 1 may be controlled to be switched to a first drainage fluid system setting, a second drainage fluid system setting and a dual-drainage fluid system setting. By way of example, when the valve core 101 is located at the water inlet connected to the first drainage fluid system 5, the dual-chamber one-way valve 1 is switched to a second drainage fluid system setting, and the second drainage fluid system 6 is connected to the water outlet of the dual-chamber one-way valve 1; when the valve core 101 is located at the water inlet connected to the second drainage fluid system 6, the dual-chamber one-way valve 1 is switched to a first drainage fluid system setting, and the first drainage fluid system 5 is connected to the water outlet of the dual-chamber one-way valve 1; when the valve core 101 is located between the two water inlets, the dual-chamber one-way valve 1 is switched to the dual-drainage fluid system setting, and both of the first drainage fluid system 5 and the second drainage fluid system 6 are connected to the water outlet of the dual-chamber one-way valve 1. With the cooperation of the valve core 101 and the valve sleeve 102, the settings of the dual-chamber one-way valve 1 may be simply switched via the rotation of the valve core 101, which is simple and convenient in operation.

In order to avoid excessive rotation of the valve core 101 of the dual-chamber one-way valve 1, a stopper 10 is arranged on one side of the water inlet of the dual-chamber one-way valve 1 close to the water outlet, so that the valve core 101 can only rotate between the stoppers 10 of the two water inlets without rotating to the water outlet, thereby avoiding the problem of excessive rotation of the valve core 101 of the dual-chamber one-way valve 1.

Considering that the proportion of the drainage fluid to be detected that needs to be collected and filtered to the total flow of the drainage fluid is relatively small, the path length of the direct-passing main path 3 is less than that of the filtering branch 2, that is to say, the direct-passing main path 3 and a pipeline connecting the dual-chamber one-way valve 1 and the switching valve 4 are positioned on a straight line to form a main pipeline, and the filtering branches 2 are branch pipelines connected to two ends of the direct-passing main path 3 in parallel. In the process of collecting and filtering the drainage fluid, only a small part of the drainage fluid to be detected needs to be filtered, by adopting the above-mentioned arrangement, most of the drainage fluid can directly flow into the fluid collection bottle 7 from the main pipeline without filtering and the drainage fluid to be detected can flow into the fluid collection bottle 7 via the branch pipeline; due to the length of the main pipeline is less than that of the branch pipeline, thus reducing the time for the whole collection process of the drainage fluid and improving the efficiency for collection and filtration of the drainage fluid.

In order to conveniently collect soft tissue debris in the filtering branch 2, the filtering branch 2 may be detachably connected in parallel to the two ends of the direct-passing main path 3 via connection joints 11. In consideration of convenience in installation, chamfers are arranged at two ends of the connection joint 11, so that when the filtering branch 2 and the direct-passing main path 3 are assembled, the filtering branch 2 can be smoothly sleeved and fixed with the connection joint 11 under the action of chamfers, and the filtering branch 2 is detachably connected to the switching valve 4 and the fluid collection bottle 7.

With regard to the structure, specifically, the filtering branch 2 may include a filtering barrel 201 and a filtering member 202 (e.g., a filter screen) arranged in the filtering barrel 201. As the drainage fluid flows through the filtering branch 2, soft tissue debris in the drainage fluid can be filtered by the foldable filtering barrel 201 and left in the foldable filtering barrel 201.

It should be noted that, in some instances, the amount of soft tissue debris in the drainage fluid may be higher, and therefore, the filtering member 202 may be a foldable filter element that suitably increases the space within the filter element to accommodate more soft tissue debris based on the amount of soft tissue debris retained therein. In addition, with the foldable filter element, soft tissue debris can be retained in the filter element rather than in the filtering barrel 201, and when the soft tissue debris is excessive, the foldable filter element can be replaced without replacing the entire filtering branch 2.

It should be noted that in practical application, the particle size of the retained sample is selected by changing the mesh size of the filtering member 202.

Sterile transparent plastic or plastic which can be sterilized by radiation, such as PVC or PVP, may be employed as the material of the above-mentioned dual-chamber suction filtering device.

In order to ensure that drainage fluid can smoothly flow in the dual-chamber suction filtering device, the diameters of the connecting pipeline of the first drainage fluid system 5 and the dual-chamber one-way valve 1, the connecting pipeline of the second drainage fluid system 6 and the dual-chamber one-way valve 1, the connecting pipeline of the dual-chamber one-way valve 1 and the switching valve 4, the pipeline of the direct-passing main path 3 and the pipeline of the filtering branch 2 should be controlled within a range of 8-12 mm, for example, about 10 mm. By comprehensively considering the fluidity of the conventional drainage fluid and the particle size of the soft tissue debris, the diameter of each pipeline is limited in the above-mentioned range, so that the drainage fluid with the soft tissue debris can smoothly flow in the dual-chamber suction filtering device, and the soft tissue debris is prevented from blocking the pipeline. It should be noted that the diameters of the above-mentioned pipelines cannot be too larger, otherwise it will not only affect the flexibility of the pipeline and be inconvenient to install, but also affect the compactness of the entire device, resulting in an excessive space occupation.

Also, the wall thickness of the pipeline should be less than or equal to 1.5 mm in consideration of flexibility, but should be greater than or equal to 0.5 mm in order to avoid breakage of the pipeline due to excessively thin wall thickness during installation.

### Example 2

This Example provides a dual-chamber suction filtering method, which comprises the following steps:

Step S1: water inlet of a dual-chamber one-way valve is connected to a first drainage fluid system and a second drainage fluid system respectively, and water outlet of a filtering branch and water outlet of a direct-passing main path are connected to a fluid collection bottle;

Step S2: when it is necessary to collect and filter the drainage fluid to be detected in the first drainage fluid system and the second drainage fluid system at the same time, the dual-chamber one-way valve is switched to a dual-drainage fluid system setting, both of the first drainage fluid system and the second drainage fluid system are connected to the water inlet of the dual-chamber one-way valve, and the switching valve is switched to a filtering setting; the drainage fluid in the first drainage fluid system and the second drainage fluid system flows through the dual-chamber one-way valve and the filtering branch in turn and then flows into the fluid collection bottle, and soft tissue debris may be filtered and retained in the filtering branch when the drainage fluid flows through the filtering branch;

when it is necessary to collect and filter the drainage fluid to be detected in the first drainage fluid system, the dual-chamber one-way valve is switched to a first drainage fluid system connection setting, and the switching valve is switched to a filtering setting; the drainage fluid in the first drainage fluid system flows through the dual-chamber one-way valve and the filtering branch in turn and then flows into the fluid collection bottle, and soft tissue debris may be filtered and retained in the filtering branch when the drainage fluid flows through the filtering branch;

when it is necessary to collect and filter the drainage fluid to be detected in the second drainage fluid system, the dual-chamber one-way valve is switched to a second drainage fluid system setting, and the switching valve is switched to a filtering setting; the drainage fluid in the second drainage fluid system flows through the dual-chamber one-way valve and the filtering branch in turn and then flows into the fluid collection bottle, and soft tissue debris may be filtered and retained in the filtering branch when the drainage fluid flows through the filtering branch;

when there is no need to collect and filter the drainage fluid in the first drainage fluid system and/or the second drainage fluid system, the dual-chamber one-way valve is switched to a dual-drainage fluid system setting and the switching valve is switched to a direct-passing setting, the drainage fluid in the first drainage fluid system and/or the second drainage fluid system flows into the fluid collection bottle via the direct-passing main path.

Compared with the prior art, the beneficial effects of the dual-chamber suction filtering method provided in this Example are basically the same as that of the dual-chamber suction filtering device provided in Example 1, which will not be repeated here.

The above description is merely a preferred embodiment of the present application, but the scope of protection of the present application is not limited thereto, and any changes or substitutions that may be readily conceived by those skilled in the art within the scope of the present disclosure are intended to be within the scope of protection of the present application.

1. A dual-chamber suction filtering device comprising a dual-chamber one-way valve, a switching valve, and a filtering branch and a direct-passing main path which are connected to each other in parallel;
wherein the water outlet of the dual-chamber one-way valve is connected to the water inlet of the filtering branch and the water inlet of the direct-passing main path;
the switching valve is provided on the joint between the water inlet of the filtering branch and the water inlet of the direct-passing main path;
the dual-chamber one-way valve has a first drainage fluid system setting, a second drainage fluid system setting and a dual-drainage fluid system setting, and the switching valve has a filtering setting and a direct-passing setting;
and the water inlet of the dual-chamber one-way valve is connected to the first drainage fluid system and/or second drainage fluid system when in use, respectively.

2. The dual-chamber suction filtering device according to claim 1, wherein the switching valve also has a fluid stop setting.

3. The dual-chamber suction filtering device according to claim 1 or 2, wherein in the dual-chamber one-way valve, an included angle connected to the water inlet of the first drainage fluid system and the water inlet of the second drainage fluid system is 60°, an included angle connected to the water inlet and the water outlet of the first drainage fluid system is 120°, and an included angle connected to the water inlet and the water outlet of the second drainage fluid system is 120°.

4. The dual-chamber suction filtering device according to claim 1, wherein a stirring member is arranged on one side of the switching valve close to the dual-chamber one-way valve.

5. The dual-chamber suction filtering device according to claim 1, wherein a spiral structure is arranged on the inner wall of the connecting pipeline between the dual-chamber one-way valve and the switching valve.

6. The dual-chamber suction filtering device according to claim 1, wherein a path length of the direct-passing main path is less than that of the filtering branch.

7. The dual-chamber suction filtering device according to claim 1, wherein the filtering branch is detachably connected in parallel to the two ends of the direct-passing main path via connection joints.

8. The dual-chamber suction filtering device according to claim 1, wherein the filtering branch comprises a filtering barrel and a filtering member arranged in the filtering barrel.

9. A dual-chamber suction filtering device according to any one of claims 1 to 8, wherein the filtering member is a collapsible filter element.

10. A dual-chamber suction filtering method, wherein by adopting the dual-chamber suction filtering device as claimed in any one of claims 1 to 9, the filtering method comprises the following steps of:
Step S1: connecting the water inlet of the dual-chamber one-way valve to the first drainage fluid system and the second drainage fluid system respectively, and connecting the water outlet of the filtering branch and the water outlet of the direct-passing main path to a fluid collection bottle;
Step S2: when it is necessary to collect and filter the drainage fluid to be detected in the first drainage fluid system and the second drainage fluid system at the same time, switching the dual-chamber one-way valve to a dual-drainage fluid system setting, connecting both of the first drainage fluid system and the second drainage fluid system to the water inlet of the dual-chamber one-way valve, and switching the switching valve to a filtering setting; wherein the drainage fluid in the first drainage fluid system and the second drainage fluid system flows through the dual-chamber one-way valve and the filtering branch in turn and then flows into the fluid collection bottle, and soft tissue debris may be filtered and retained in the filtering branch when the drainage fluid flows through the filtering branch;
when it is necessary to collect and filter the drainage fluid to be detected in the first drainage fluid system, switching the dual-chamber one-way valve to a first drainage fluid system connection setting, and switching the switching valve to a filtering setting; wherein the drainage fluid in the first drainage fluid system flows through the dual-chamber one-way valve and the filtering branch in turn and then flows into the fluid collection bottle, and soft tissue debris may be filtered and retained in the filtering branch when the drainage fluid flows through the filtering branch;
when it is necessary to collect and filter the drainage fluid to be detected in the second drainage fluid system, switching the dual-chamber one-way valve to a second drainage fluid system setting, and switching the switching valve to a filtering setting; wherein the drainage fluid in the second drainage fluid system flows through the dual-chamber one-way valve and the filtering branch in turn and then flows into the fluid collection bottle, and soft tissue debris may be filtered and retained in the filtering branch when the drainage fluid flows through the filtering branch;
when there is no need to collect and filter the drainage fluid in the first drainage fluid system and/or the second drainage fluid system, switching the dual-chamber one-way valve to a dual-drainage fluid system setting and switching the switching valve to a direct-passing setting, wherein the drainage fluid in the first drainage fluid system and/or the second drainage fluid system flows into the fluid collection bottle via the direct-passing main path.
